(19) United States
(12) Patent Application Publication
(10) Pub. No.: US 2013/0079726 A1
(43) Pub. Date: Mar. 28, 2013

(54) SYRINGES

(71) Applicant: Graham John Madin, Mielkendorf (DE)

(72) Inventor: Graham John Madin, Mielkendorf (DE)

(21) Appl. No.: 13/516,478

(22) PCT Filed: Dec. 13, 2010
(86) PCT No.: PCT/GB10/52078

§ 371 Date: Sep. 12, 2012

(30) Foreign Application Priority Data
Dec. 16, 2009 (GB) 0921939.5
May 12, 2010 (GB) 1007934.1

(51) Int. Cl. A61M 5/32; A61M 5/34

(52) U.S. Cl. CPC A61M 5/3273; A61M 5/34; A61M 5/347
USPC 604/192; 604/240; 604/241

(57) ABSTRACT

A syringe for delivering medication through an intravenous port, or for providing a combination of medications includes a barrel with a plunger in slidable and sealing engagement therein, and a piercing member formed integrally with the barrel. The piercing member includes a bore in communication with the distal end of the barrel, and a piercing point and an aperture at a distal end of the piercing member. The distal end of the barrel sealingly connects to a transport cap, an intravenous port or an injection needle. The distal end of the piercing member is removable and includes an external recess and a corresponding internal step to define a separation plane for the distal end of the piercing member. The piercing member is used to draw up one or more medications into the barrel, and is then removed to enable the transport cap to be attached to seal the contents.

The present invention relates to syringes, and in particular to syringes for delivering medication through an intravenous port, or for providing a combination of medications.

A patient who requires regular intravenous medication is normally fitted with an intravenous (IV) port, to avoid the need for separate injections. Often only one medication is required, and this is delivered by means of a standard syringe having a plunger working in a barrel. Initially a standard metal needle is attached to the syringe. The needle is inserted through a rubber sealing septum into a vial containing the medication. The correct amount of medication is drawn into the barrel, and the needle removed from the vial. For some types of IV port, the needle must be removed from the syringe before the syringe can be connected to the IV port. This carries a risk of needlestick injury to the health care worker performing the procedure. Some types of IV port accept a needle, but there is still a risk of needlestick injury both when the syringe is attached to, and removed from the port.

It is also quite common for a patient (especially in hospital) to require injections containing several different medications, perhaps because the patient has a complicated condition or several different conditions. In such a case it is advantageous, where possible, to combine different medications in one injection, to minimise the number of injections that are needed.

Combining of medications is known as compounding and is carried out to fit the unique medical needs of the patient. For example, it may be required to obtain an exact dose, to change the form of medication from solid to liquid, or to avoid a non-essential component that the patient is allergic to. The compounded medication may be administered by a standard syringe, or through an IV port.

Where such multiple-medication injections are to be given, a syringe is filled with the correct amounts of the different medications in the hospital pharmacy or ward, or by an external compounding company. Again, a standard syringe having a plunger working in a barrel, to which is attached a metal needle, is used for this purpose. As described above, the needle is inserted through a rubber sealing septum into a vial containing a medication. The correct amount of the medication is drawn into the barrel, the needle removed from the vial, and the process repeated until the barrel contains all the necessary medications. It may be possible to give the injection using the same needle, but if there is a risk of contamination while the syringe is being taken to the patient, the syringe must be capped. As re-capping a needle is against all guidelines for injection safety, because of the relatively high risk of needlestick injury, the original needle is removed and discarded. A new, covered needle is attached for the injection. Obviously the removal of the needle still carries the risk of a needlestick injury.

If the compounded medication is for delivery through an IV port, the original needle is removed. It is likely then that a transport cap will be attached, to prevent contamination while the syringe is being taken to the patient. The transport cap will be removed so that the syringe can be attached to the IV port either directly or with a further needle if required. Whether or not a transport cap is attached, the removal of the original needle carries a risk of a needlestick injury.

One solution to the problem of needlestick is for a special plastics pin, rather than the standard metal needle, to be attached to the syringe for drawing up the medication or medications. When the syringe is filled the pin is removed, and a transport cap put on. This is in turn removed for IV delivery, or replaced by a needle for injection. The special pins are generally used only by external compounding companies, as they are very expensive. It is not generally practical to use them in a hospital environment, particularly for single medication for IV delivery, because of the cost.

A syringe shown in WO2006/029528 aims to reduce the risk of needlestick by using a cannula moulded integrally with the barrel. The cannula is able to pierce the septum of a vial to allow contents to be drawn into the barrel. The cannula may then be used to deliver the contents to an intravenous line. Alternatively, it has a weakened portion on either the external or internal surface to enable the end of the cannula to be broken off and a metal needle fitted for injection. The barrel and cannula may be provided with a luer connection for the needle. The disadvantage of this construction is that considerable force must be applied to break off the end, and it may not break cleanly, with a risk that plastics particles could enter the barrel. Further, although the cannula may be covered with a standard cap, there is no indication of how the filled syringe is secured for transportation to reduce the risk of contamination.

According to the present invention, we provide a syringe comprising a barrel having a plunger in slidable and sealing engagement therein, and a piercing member formed integrally with the barrel, the piercing member having a bore in communication with the distal end of the barrel, and a piercing point and an aperture at its distal end, and the distal end of the barrel also having a means for sealing connection of a transport cap, an intravenous port or an injection needle, the distal end of the piercing member being removable and having an external recess and a corresponding internal step to define a separation plane for the distal end.

The invention enables the medications to be drawn up without a needle, so that the risk of needlestick injury on removal of the needle is avoided. In use, the distal end of the piercing member is removed when the syringe has been filled. This operation does not carry a needlestick risk and reduces the possibility of contamination. If the medication can be delivered immediately to the patient via an IV port, the syringe may be attached to the IV port straight away. If the syringe requires transportation to the patient, the transport cap is attached to seal the contents. For injection, the transport cap is removed and a standard needle attached. For delivery through an IV port, the transport cap is removed and the syringe attached to the IV port. The arrangement of the external recess and internal step enables the distal end to be removed with a minimum of force, while ensuring a clean break, to reduce the risk of plastics particles entering the barrel, and contaminating the medication. Thus, a single, simple item can readily be used without risk of needlestick injury or contamination, for IV delivery of a single medication, as well as for compounded medications either through IV delivery at a needle-free port or injection.

Conveniently, the distal end of the piercing member is removed by application of a substantially lateral force.

A separate protective cap may be provided to protect the piercing member initially. The protective cap will be removed to enable medications to be drawn up, and may then be used to apply the force to remove the distal end of the piercing member. The cap may be constructed with an open-ended removal recess at its distal end, separated from the recess at its proximal end which fits over the piercing member for protection. The removal recess at the distal end fits over the distal end of the piercing member. The cap may then be used to apply the force to remove the distal end of the piercing member and to retain the removed end in the removal recess for disposal. The removal recess is conveniently shaped to conform to the profile of the distal end of the piercing member so that removal and retention are assured. The distal end of the cap may be provided with an enlarged portion for ease of use. The enlarged portion may include a funnel leading to the removal recess, to provide a guide for the removable distal end of the piercing member. The funnel may also have one or more guide ribs.

After the distal end of the piercing member has been removed, the transport cap may be attached to seal the contents.

The means for connecting the transport cap, the IV port or the injection needle may be a luer lock connection. The distal end of the barrel may then be provided with a hollow cylindrical collar surrounding the piercing member and provided with an internal screw thread of standard luer lock form. The barrel may therefore be connected to any other device having an appropriate standard external luer lock thread. The transport cap is provided with a corresponding male thread, in the same way as a standard luer lock needle. The protective cap may also be provided with a corresponding male thread, or with projections for engagement with the screw thread.

The piercing member is arranged so that after removal of its distal end its proximal end remains on the barrel, and acts as a support hub for the interior of the transport cap, the IV port or the needle.

The piercing member conveniently has at least one, and preferably two apertures at its distal end.

The syringe barrel and piercing member may be moulded in one piece from a suitable plastics material. One such material is polycarbonate, which has sufficient rigidity to make the piercing point able to pierce rubber septums and the like, and will be easy to snap off. The point will not be suitable for piercing the skin of a human or animal body at all.

The one-piece moulding preferably also includes the hollow cylindrical collar. This makes manufacture of the barrel particularly easy and inexpensive. The protective cap and the transport cap must, of course, be separate components. They may also be moulded from polycarbonate, but any suitable plastics material could be used. In particular, the protective cap may be of polypropylene.

The plunger is preferably of a standard construction, .comprising a rod carrying a head, on which is mounted a seal for engagement with the internal surface of the barrel.

The syringe as a whole therefore has few components, and so is simple to manufacture and assemble.

An embodiment of the invention is illustrated, by way of example only, in the accompanying drawings, in which:

FIG. 1 is a perspective view of a syringe according to the invention;

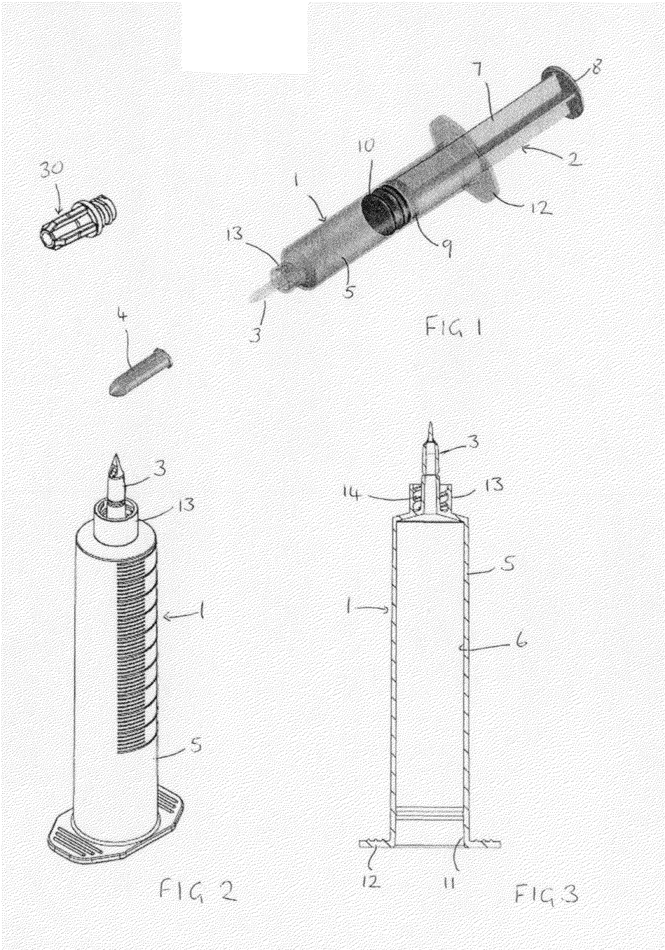

FIG. 2 is a perspective view of the barrel of the syringe of FIG. 1;

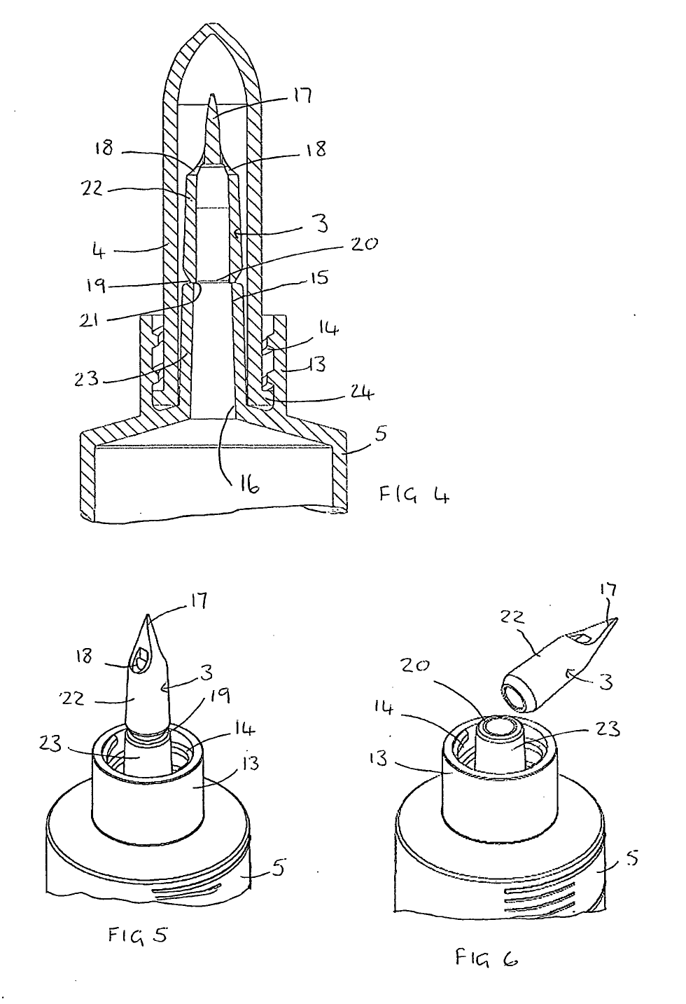

FIG. 3 is a longitudinal section through the barrel of FIG. 2;

FIG. 4 is an enlarged view of a longitudinal section through the distal end of the barrel of FIG. 2 and including the protective cap;

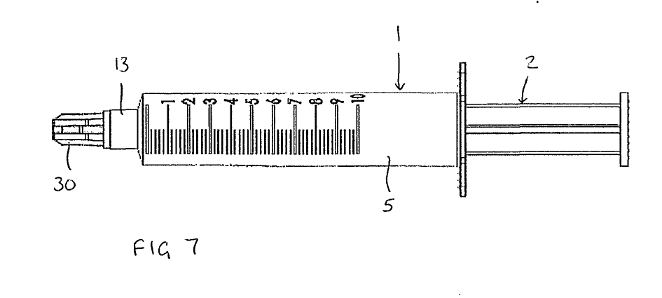

FIG. 5 is an enlarged perspective view of the distal end of the barrel of FIG. 2;

FIG. 6 is similar to FIG. 5, but with the piercing member removed;

FIG. 7 shows the syringe with the transport cap in place;

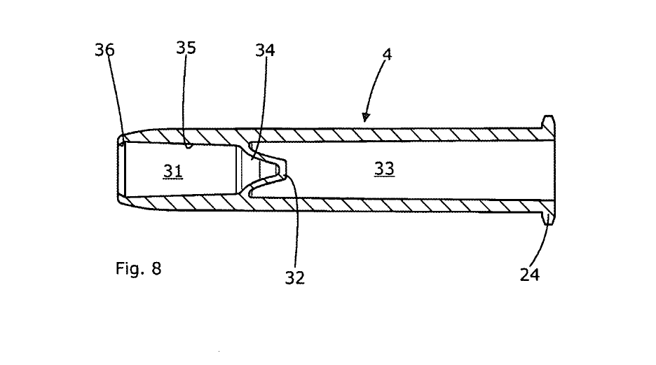

FIG. 8 is a cross-section through a modified protective cap;

FIG. 9 is a cross-section through another embodiment of protective cap;

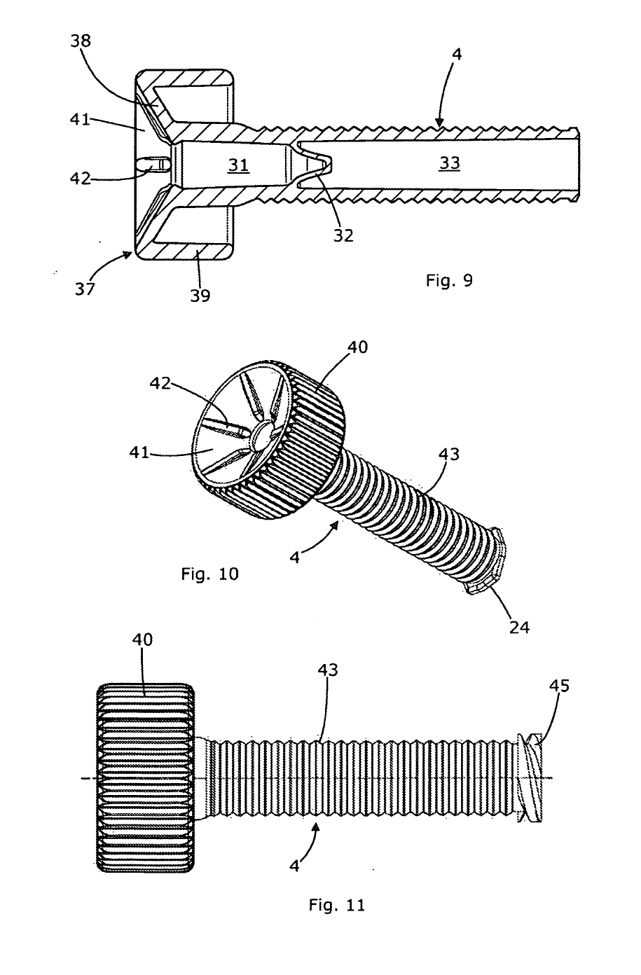

FIG. 10 is a perspective view of the cap of FIG. 9; and

FIG. 11 is a side view of a protective cap showing a modification of the cap of FIGS. 9 and 10.

The syringe shown in the Figures comprises a barrel 1 in which works a plunger 2, and a piercing member 3 formed integrally with the barrel 1. A protective cap 4 is provided as a separate component, as is a transport cap 30.

The barrel 1 is generally cylindrical, having a cylinder 5 for containing a liquid to be injected, with an inner cylindrical surface 6 in which the plunger 2 slides sealingly. The outer surface has markings indicating the volume contained. The plunger 2 is of known construction, having a rod 7 with a finger plate 8 at its proximal end and at its distal end a head 9 to which is mounted an elastomeric seal 10 forming a complete seal with the inner surface 6 of the barrel 1.

The proximal end 11 of the barrel 1 is open, and is provided with a gripping flange 12. The distal end is provided with the piercing member 3, and a hollow cylindrical collar 13 also formed integrally with the barrel 1. The collar 13 surrounds the proximal end of the piercing member 3, and is spaced from it. The internal surface of the collar 13 is provided with a female screw thread 14, forming a standard luer lock arrangement, and allowing a sealing connection of the transport cap 30 (as shown in FIG. 7) or a standard metal injection needle (not shown).

As best seen in FIGS. 4 to 6, the piercing member 3 is a substantially cylindrical member having a conical blind bore 15 whose open proximal end 16 is in communication with the interior of the barrel 1. At its closed distal end the member 3 has a solid sharp piercing point 17 of reduced diameter, and a pair of opposed liquid inlet apertures 18 at the base of the point 17. The apertures 18 lead to the interior of the bore 15, at its closed end, and are arranged to allow flow of liquid axially along the side of the point 17 and radially directly into the bore 15. Adjacent its proximal end 16, and at a point spaced distally from the free end of the collar 13, the piercing member 3 has an external shaped recess 19 to reduce the thickness of the wall of the member 3. There is also a small reduction in diameter of the bore 15, so that the step 21 in the bore 15 at that point corresponds to and is opposite the recess 19. The recess 19 and the step 21 define a separation plane at which the distal end snaps off when a lateral force is applied. The shape of the recess 19 and the step 21 define an intrinsic snapping angle between the distal portion 22 and the remaining proximal portion 23. The line 20 shows where the distal and proximal portions separate, with the distal portion 22 of the piercing member 3 being readily removed in this way. The arrangement of the recess 19 and the step 21 is such that the wall of the cap has a relatively small radial thickness at the separation plane, so that a relatively small force is required to snap the distal portion 22 off with a clean break to reduce the risk of plastics particles being generated and falling into the barrel 1. The remaining portion 23 of the piercing member 3 projects slightly from the collar 13, and forms a hub for supporting the transport cap 30, an intravenous (IV) port (not shown) or a metal needle (not shown) which may then be sealingly attached by the screw thread 14. As shown in FIG. 4, the protective cap 4 has projections 24 which engage in the screw thread 14, in the same way as a luer lock needle. The protective cap 4 is attached with the piercing member 3 in place, to protect it until the syringe is used.

The transport cap 30 has a male screw thread corresponding to the female screw thread 14, so that the cap 30 engages sealingly with the syringe.

The barrel 1, piercing member 3 and collar 13 are moulded in one piece, from polycarbonate. Polycarbonate can be moulded with sufficient rigidity to make the sharp piercing point 17, and will also enable the distal portion 22 to be snapped off easily. The point 17 will not be suitable for piercing skin, so cannot be used for direct injection into the human or animal body. It will however be able to pierce the rubber septums commonly used to seal vials of liquid medications. The protective cap 4 and transport cap 30 may also be of polycarbonate, although the protective cap 4 may instead be of polypropylene.

The barrel 1 is assembled with the plunger 2, and a protective cap 4, which is attached mechanically during the assembly process. Before use, the syringe is as shown in FIG. 4, therefore. For use, the protective cap 4 is removed, and the piercing point 17 is used to pierce the seal of a vial containing a liquid medication. The plunger 2 is withdrawn to draw up a given amount of the medication, through the apertures 18 into the bore 15 and then into the cylinder 5. The piercing member 3 is then removed from the vial. If a compounded medication is required, the process is repeated with another medication, until all the necessary medications are present in the cylinder 5. The protective cap 4 is then used to apply a lateral force to the distal end of the piercing member 3, to snap off the distal portion 22 along the line 20, as shown in FIG. 6. The transport cap 30 is then screwed on to the barrel 1 to seal the contents. The filled syringe is transported to the patient, where the transport cap 30 is removed and a standard luer lock metal needle attached instead, to enable the patient to be injected. Alternatively, if the medication is to be delivered through an IV port, the transport cap 30 is removed and the syringe attached to the IV port. In either case the syringe may then be disposed of safely.

If a single medication is being delivered through an IV port, the filling of the syringe may take place by the patient. In that event, after removal of the distal portion 22 of the piercing member 3, the syringe may be attached immediately to the IV port, and the transport cap 30 is not required.

FIGS. 8 to 11 show modifications of the protective cap 4 which enable it to be used to remove and retain the removable distal portion 22 of the piercing member 3, and corresponding reference numerals have been applied to corresponding parts.

In FIG. 8, the protective cap 4 has a greater axial length than the cap of FIG. 4. At its distal end the cap has an open-ended removal recess 31, separated by an integral wall 32 from the proximal recess 33 which is placed over the piercing member 3 initially. The wall 32 ensures that the protective function of the cap 4 for the piercing member 3 is maintained.

The removal recess 31 is shaped to conform to the profile of the removable distal portion 22 of the piercing member 3. Thus, the recess 31 has a reduced diameter substantially conical portion 34 corresponding to the point 17, leading to a bore 35 whose diameter increases towards the open end, to correspond to the increasing diameter portion from the point 17 to the recess 19. At the open end the bore diameter is reduced again so the end 36 corresponds to the shape of the recess 19. Otherwise the cap of FIG. 8 is the same as that of FIG. 4, with the projections 24 which are received in the collar 13.

In use, the cap 4 of FIG. 8 is unscrewed and removed to enable the medications to be drawn into the barrel 1 through the piercing member 3. At the end of that process, for removal of the distal portion 22, the cap 4 is reversed, and the removal recess 31 placed over the piercing member 3, with the distal portion 22 being inserted into the recess 31 until the reduced diameter end 36 snaps into the recess 19 to capture the distal portion 22 in the recess 31. A lateral force may then be applied to the cap 4 to remove the distal portion 22. The distal portion 22 is retained in the recess 31 so that the two can be disposed of together. This, of course, means that the distal portion, which is quite small, does not have to be disposed of separately, and there is no danger of it being lost.

FIGS. 9 and 10 show a modification of the cap 4 of FIG. 8. In FIGS. 9 and 10 the cap has an enlarged portion 37 at the distal end, comprising a frusto-conical portion 38 extending radially outwardly and distally, and an axially-extending return 39 which terminates approximately mid-way along the length of the recess 31. The return 39 has axial knurling 40 to make it easy to grasp. The frusto-conical portion 38 forms a funnel 41 for guiding the point 17 of the piercing member 3 into the recess 31. The funnel 41 also acts to protect the user's fingers as the cap is placed over the point 17. The funnel 41 has radial ribs 42 (six are shown, but any suitable number could be provided) to help prevent the point 17 from slipping out of the funnel 41.

The outer surface of the main part of the cap 4 is provided with a circumferential grip profile 43. This, together with the knurling 40, aids in unscrewing the cap 4 and in placing it over the piercing member 3 for removal of the distal portion 22.

The construction of the cap of FIGS. 9 and 10 is otherwise the same as that of FIG. 8. The operation is also the same, but with the features of FIGS. 9 and 10 providing advantages for ease of use.

FIG. 11 shows a cap 4 very similar to that of FIGS. 9 and 10. In FIG. 11 the projections 24 at the proximal end of the cap have been replaced by a twin-start male thread 45 corresponding to the female thread 14 on the collar 13. This enables the cap 4 to be put on and removed with a single turn, rather than the two required for the projections 24. The construction and operation of the cap of FIG. 11 is otherwise the same as that for FIGS. 9 and 10.

In any of the embodiments the luer lock arrangement on the collar 13 may be replaced with a standard luer slip arrangement, where the connection is made through corresponding conical surfaces.

The syringe in all its embodiments is easy and inexpensive to manufacture, because it has few components, but it provides the ability to fill a syringe with different liquid medications in a simple and efficient way, while reducing the risk of needlestick injury and contamination. It is particularly useful where delivery of the medication is through an IV port, as no needle at all is required, either for filling the syringe or delivery through the IV port. The whole process is therefore needle-free.

1. A syringe comprising:
a barrel having a plunger in slidable and sealing engagement therein; and
a piercing member formed integrally with the barrel, the piercing member including:
a bore in communication with a distal end of the barrel, and
a piercing point and an aperture at a distal end of the piercing member,
wherein the distal end of the barrel includes an arrangement for a sealing connection of a transport cap, an intravenous port or an injection needle, and
wherein the distal end of the piercing member is removable and includes an external recess and a corresponding internal step to define a separation plane for the distal end of said piercing member.

2. A syringe as claimed in claim 1, wherein the distal end of the piercing member is removable by application of a substantially lateral force.

3. A syringe as claimed in claim 1, wherein the syringe includes a separate protective cap for initial protection of the piercing member.

4. A syringe as claimed in claim 3, wherein the protective cap is removed to enable medications to be drawn up, and is then used to apply the force to remove the distal end of the piercing member.

5. A syringe as claimed in claim 4, wherein the protective cap includes an open-ended removal recess at a distal end thereof, separated from a recess at a proximal end thereof which fits over the piercing member for protection.

6. A syringe as claimed in claim 5, wherein the removal recess at the distal end of the protective cap fits over the distal end of the piercing member.

7. A syringe as claimed in claim 6, wherein the protective cap is used to apply the force to remove the distal end of the piercing member and to retain the removed distal end in the removal recess for disposal.

8. A syringe as claimed in claim 6, wherein the removal recess is shaped to conform to a profile of the distal end of the piercing member.

9. A syringe as claimed in claim 3, wherein the distal end of the cap includes an enlarged portion.

10. A syringe as claimed in claim 9, wherein the enlarged portion includes a funnel leading to the removal recess to provide a guide for the removable distal end of the piercing member.

11. A syringe as claimed in claim 10, wherein the funnel at least one guide rib.

12. A syringe as claimed in claim 1, wherein the arrangement for connecting the transport cap, the intravenous port or the injection needle includes a luer lock connection.

13. A syringe as claimed in claim 12, wherein the distal end of the barrel includes a hollow cylindrical collar surrounding the piercing member and including an internal screw thread of standard luer lock form.

14. A syringe as claimed in claim 13, wherein the transport cap includes a corresponding male screw thread of standard luer lock form.

15. A syringe as claimed in claim 12, wherein the protective cap includes a corresponding male screw thread of standard luer lock form.

16. A syringe as claimed in claim 12, wherein the protective cap includes projections that engage with the internal screw thread.

17. A syringe as claimed claim 1, wherein the piercing member is arranged so that after removal of the distal end thereof, a proximal end thereof remains on the barrel and acts as a support hub for an interior of the transport cap, the intravenous port or the needle.

18. A syringe as claimed in claim 1, wherein the piercing member includes two apertures at the distal end thereof.

19. A syringe as claimed in claim 1, wherein the syringe barrel and piercing member are moulded in one piece from a plastic material.

20. A syringe as claimed in claim 19, wherein the distal end of said barrel includes a hollow cylindrical collar surrounding the piercing member and the one-piece moulding includes the hollow cylindrical collar.

21. A syringe comprising:
a barrel having a plunger in slidable and sealing engagement therein; and
a piercing member formed integrally with said barrel, said piercing member including a bore in communication with a distal end of said barrel, and a piercing point and an aperture at a distal end of the piercing member,
wherein said distal end of said barrel includes an arrangement for sealing connection of a transport cap, an intravenous port or an injection needle,
wherein said distal end of said piercing member is removable by a protective cap separate from said transport cap, said protective cap including a recess at a proximal end thereof which fits over said piercing member for initial protection and an open-ended removal recess at a distal end thereof, separated from said recess at said proximal end, said removal recess at said distal end of said protective cap fitting over said distal end of said piercing member, said protective cap being removable to enable medications to be drawn up, and
wherein said distal end of said piercing member being insertable into said removal recess, said protective cap then configured to apply a force to remove said distal end of said piercing member.

22. A syringe comprising:
a barrel including a plunger in slidable and sealing engagement therein; and
a piercing member formed integrally with said barrel, said piercing member including a bore in communication with a distal end of said barrel, and a piercing point and an aperture at a distal end of the piercing member,
wherein said distal end of said barrel includes an arrangement for sealing connection of a transport cap, an intravenous port or an injection needle,
wherein said distal end of said piercing member is removable, and
wherein the piercing member includes a separate protective cap for initial protection, a distal end of the protective cap including an enlarged portion.
